(19) United States
(12) Patent Application Publication
(10) Pub. No.: US 2010/0184996 A1
(43) Pub. Date: Jul. 22, 2010

(54) PROCESS OF AMIDE FORMATION

(75) Inventors: Alcide Perboni, Verona (IT); Nicola Giubellina, Verona (IT)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED, Middlesex, EN (GB)

(21) Appl. No.: 12/663,919

(22) PCT Filed: Jun. 18, 2008
(86) PCT No.: PCT/EP2008/057642

§ 371 Date: Dec. 10, 2009

(30) Foreign Application Priority Data
Jun. 20, 2007 (GB) 0711974.6
Jun. 20, 2007 (GB) 0711976.1

(51) Int. Cl. C07D 207/04

(52) U.S. Cl. 548/568; 548/571; 548/569

(57) ABSTRACT

A process for the formation of 2-(methyloxy)-N-[2-methyl-1-phenyl-2-(1-pyrrolidinyl)propyl]-4,6-bis(trifluoromethyl)benzamide is disclosed, comprising treatment of 2,4-ditrifluoromethyl-6-methoxy-benzoic acid with an arylsulphonyl halide, C₁₋₆alkylsulphonyl halide or di-C₁₋₆alkylphosphoryl chloride, in the presence of a base and an aprotic solvent; followed by reaction with [2-methyl-1-phenyl-2-(1-pyrrolidinyl)propyl]amine or a salt thereof. Also disclosed is a process for the formation of [2-methyl-1-phenyl-2-(1-pyrrolidinyl)propyl]amine comprising: i) treatment of an α-haloketone with an alcohol in the presence of a base followed by reaction with pyrrolidine; ii) treatment of the product of stage i) with Ar—CH(R⁵)N H₂ wherein R⁵ is C₁₋₄alkyl and Ar is optionally substituted phenyl, followed by reduction with a sodium borohydride derivative; and iii) reduction of the product of stage ii) with hydrogen and a palladium catalyst. Also disclosed is the novel compound 2-pyrrolidinyl-2-methylpropiophenone or a salt or solvate thereof.

The present invention provides a one stage procedure for the formation of amides from carboxylic acids and amines, using inexpensive activating agents resulting in high yield and purity of product.

One compound for which the present invention may be used is 2-(methyloxy)-N-[2-methyl-1-phenyl-2-(1-pyrrolidinyl)propyl]-4,6-bis(trifluoromethyl)benzamide:

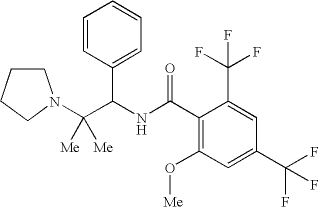

2-(Methyloxy)-N-[2-methyl-1-phenyl-2-(1-pyrrolidinyl)propyl]-4,6-bis(trifluoromethyl)benzamide and its hydrochloride salt are disclosed in WO2006067423 as being glycine transport inhibitors and useful in the manufacture of medicaments for treating neurological and neuropsychiatric disorders, in particular psychoses, dementia or attention deficit disorder. WO2006067423 discloses the preparation of this compound by reacting 2,4-ditrifluoromethyl-6-methoxy-benzoic acid and chiral [2-methyl-1-phenyl-2-(1-pyrrolidinyl)propyl]amine in an appropriate solvent such as DMF. However, the use of TBTU (2-(1H-benzotriazol-1-yl)-1,1,3,3-tetramethyluronium tetrafluoroborate) as an activating agent can produce a product that is potentially explosive, and requires special treatment.

Thus, in a first aspect, the present invention provides a process for the preparation of 2-(methyloxy)-N-[2-methyl-1-phenyl-2-(1-pyrrolidinyl)propyl]-4,6-bis(trifluoromethyl)benzamide, comprising:

step (i): treatment of 2,4-ditrifluoromethyl-6-methoxy-benzoic acid with a compound of formula (III):

R₁—X (III)

wherein R₁ is selected from the group consisting of C₁₋₆alkylsulfonyl, arylsulphonyl and diC₁₋₆alkylphosphate diester; and X is chlorine or bromine, in the presence of a base and an aprotic solvent; followed by

step (ii): reaction with [2-methyl-1-phenyl-2-(1-pyrrolidinyl)propyl]amine or a salt thereof.

As used herein, the term “C₁₋₆alkyl” refers to a straight or branched alkyl group containing 1-6 carbon atoms in all isomeric forms. Examples include, but are not limited to, methyl, ethyl, propyl, isopropyl, butyl, isobutyl, sec-butyl, tert-butyl, pentyl and hexyl. The term “C₁₋₄alkyl” refers to a straight or branched alkyl group containing 1-4 carbon atoms in all isomeric forms. Examples include methyl, ethyl, propyl, isopropyl, butyl, isobutyl, sec-butyl, and tert-butyl.

The term “aryl” as used herein refers to a phenyl or a naphthyl group, both optionally substituted with 1, 2 or 3 groups selected from: C₁₋₄alkyl, C₁₋₄alkoxy, haloC₁₋₄alkyl, C₃₋₆cycloalkyl, C₁₋₄alkoxyC₁₋₄alkyl, and CONR^{a}R^{b} (wherein R^{a} and R^{b} are independently selected from H and C₁₋₄alkyl, or R^{a} and R^{b}, together with the nitrogen atom to which they are attached, form a 4- to 7-membered ring).

As used herein, the term “alkoxy” refers to the group —O-alkyl wherein alkyl is as defined above.

As used herein, the terms “halogen” and its abbreviations “hal” or “halo” refer to fluorine, chlorine, bromine, or iodine.

As used herein, the term “haloC₁₋₄alkyl” refers to a C₁₋₄alkyl group as defined above which is substituted with 1 or more fluorine, chlorine, bromine, or iodine atoms, including with mixtures of those atoms. A haloalkyl group may, for example contain 1, 2 or 3 halogen atoms. For example, a haloalkyl group may have all hydrogen atoms replaced with halogen atoms. Examples of haloalkyl groups include, but are not limited to, fluoromethyl, difluoromethyl and trifluoromethyl.

As used herein the term “C₃₋₆cycloalkyl” refers to a saturated monocyclic hydrocarbon ring of 3 to 6 carbon atoms. Examples of such groups include cyclopropyl, cyclobutyl, cyclopentyl, or cyclohexyl and the like.

The term “C₅₋₁₀aryl” as used herein refers to a mono- or bicyclic aromatic carbocyclic group containing 5-10 carbon atoms.

As used herein, the term “carbocyclic ring” refers to a cycloalkyl or heterocyclic ring.

As used herein, the term “heterocyclic ring” refers to a monocyclic ring of the stated size which may be saturated or partially unsaturated, containing 1 nitrogen atom. Examples of such monocyclic rings include azetadinyl, pyrrolidinyl, piperidinyl, azapinyl and the like.

The compound of formula (III) may be any such compound available commercially (suppliers include Sigma-Aldrich, Alfa Aesar, TCI Organic Chemicals, Kessler Chemical, Inc., Acros Organics) or synthesised from published synthetic routes (for example Zeitschrift fur Naturforschung, B: Chemical Sciences, 42(12), 1591-4; 1987) or synthesised using standard synthetic chemistry.

In one embodiment, R₁ is an arylsulphonyl group.

In one embodiment, R₁ is selected from the group consisting of mesyl, tosyl and diethyl phosphate diester.

In one embodiment, R₁ is n-propylsulphonyl.

In one embodiment, X is chlorine.

In one embodiment, the compound of formula (III) is mesyl chloride, tosyl chloride or diethyl chlorophosphate.

In one embodiment, the compound of formula (III) is n-propylsulphonyl chloride.

In one embodiment, the base in step (i) of the process is a tertiary amine. In a further embodiment, the base in step (i) of the process is triethylamine.

In one embodiment, the aprotic solvent in step (i) of the process is selected from the group consisting of acetonitrile, methylene chloride and ethyl acetate. In one embodiment, the solvent in step (i) of the process is acetonitrile. In one embodiment, the solvent in step (i) of the process is ethyl acetate.

2-(Methyloxy)-N-[2-methyl-1-phenyl-2-(1-pyrrolidinyl)propyl]-4,6-bis(trifluoromethyl)benzamide is a chiral molecule. The (+) form and the (−) may be prepared by stereospecific synthesis and/or by resolution of the final product.

In one embodiment, in step (ii), (R)-(+)-[2-methyl-1-phenyl-2-(1-pyrrolidinyl)propyl]amine or a salt thereof is used and the final product is (R)-2-(methyloxy)-N-[2-methyl-1-phenyl-2-(1-pyrrolidinyl)propyl]-4,6-bis(trifluoromethyl)benzamide.

In one embodiment, the process gives (R)-2-(methyloxy)-N-[2-methyl-1-phenyl-2-(1-pyrrolidinyl)propyl]-4,6-bis(trifluoromethyl)benzamide with at least 90% enantiomeric excess. In one embodiment, the process gives (R)-2-(methyloxy)-N-[2-methyl-1-phenyl-2-(1-pyrrolidinyl)propyl]-4,6-bis(trifluoromethyl)benzamide with at least 95% enantiomeric excess. In one embodiment, the process gives (R)-2-(methyloxy)-N-[2-methyl-1-phenyl-2-(1-pyrrolidinyl)propyl]-4,6-bis(trifluoromethyl)benzamide with at least 99% enantiomeric excess.

In a further embodiment, the present invention provides a process for the preparation of (R)-2-(methyloxy)-N-[2-methyl-1-phenyl-2-(1-pyrrolidinyl)propyl]-4,6-bis(trifluoromethyl)benzamide, comprising:

step (i): treatment of 2,4-ditrifluoromethyl-6-methoxy-benzoic acid with a compound of formula (III):

R₁—X (III)

wherein R₁ is selected from the group consisting of C₁₋₆alkylsulfonyl, arylsulphonyl and diC₁₋₆alkylphosphate diester; and X is chlorine or bromine in the presence of a base and an aprotic solvent, followed by

step (ii): reaction with (R)-(+)-[2-methyl-1-phenyl-2-(1-pyrrolidinyl)propyl]amine or a salt thereof.

In one embodiment, the present invention provides a process for the preparation of (R)-2-(methyloxy)-N-[2-methyl-1-phenyl-2-(1-pyrrolidinyl)propyl]-4,6-bis(trifluoromethyl)benzamide, comprising:

step (i): treatment of 2,4-ditrifluoromethyl-6-methoxy-benzoic acid with a compound selected from the group consisting of mesylchloride, tosylchloride and diethylchlorophosphate in the presence of a base and an aprotic solvent, followed by

step (ii): reaction with (R)-(+)-[2-methyl-1-phenyl-2-(1-pyrrolidinyl)propyl]amine or a salt thereof.

In one embodiment, the base in step (i) of the process is a tertiary amine. In a further embodiment, the base in step (i) of the process is triethylamine.

In one embodiment, the aprotic solvent in step (i) of the process is selected from the group consisting of acetonitrile, methylene chloride and ethyl acetate. In a further embodiment, the solvent in step (i) of the process is acetonitrile.

In one embodiment, the present invention provides a process for the preparation of (R)-2-(methyloxy)-N-[2-methyl-1-phenyl-2-(1-pyrrolidinyl)propyl]-4,6-bis(trifluoromethyl)benzamide, comprising:

step (i): treatment of 2,4-ditrifluoromethyl-6-methoxy-benzoic acid with n-propylsulphonylchloride in the presence of a base and an aprotic solvent, followed by

step (ii): reaction with (R)-(+)-[2-methyl-1-phenyl-2-(1-pyrrolidinyl)propyl]amine or a salt thereof.

In one embodiment, the base in step (i) of the process is a tertiary amine. In a further embodiment, the base in step (i) of the process is triethylamine.

In one embodiment, the aprotic solvent in step (i) of the process is selected from the group consisting of acetonitrile, methylene chloride and ethyl acetate. In a further embodiment, the solvent in step (i) of the process is ethyl acetate.

In one embodiment, the present invention provides a process for the preparation of (R)-2-(methyloxy)-N-[2-methyl-1-phenyl-2-(1-pyrrolidinyl)propyl]-4,6-bis(trifluoromethyl)benzamide, comprising:

step (i): treatment of 2,4-ditrifluoromethyl-6-methoxy-benzoic acid with n-propylsulphonylchloride in the presence of triethylamine and ethyl acetate, followed by

step (ii): reaction with (R)-(+)-[2-methyl-1-phenyl-2-(1-pyrrolidinyl)propyl]amine or a salt thereof.

WO2006067423 discloses a preparation of 2-(methyloxy)-N-[2-methyl-1-phenyl-2-(1-pyrrolidinyl)propyl]-4,6-bis(trifluoromethyl)benzamide by reacting 2,4-ditrifluoromethyl-6-methoxy-benzoic acid and chiral [2-methyl-1-phenyl-2-(1-pyrrolidinyl)propyl]amine, as shown below:

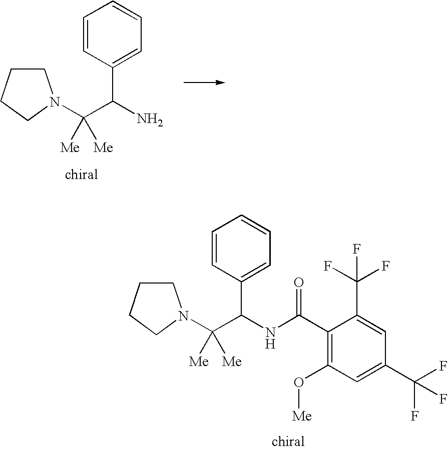

The formation of the chiral diamine intermediate [2-methyl-1-phenyl-2-(1-pyrrolidinyl)propyl]amine disclosed in WO2006067423 involves the use of 2-methyl-2-(1-pyrrolidinyl)propanenitrile as a starting material, which is itself synthesised from pyrrolidine using potassium cyanide and phenyllithium, as shown below:

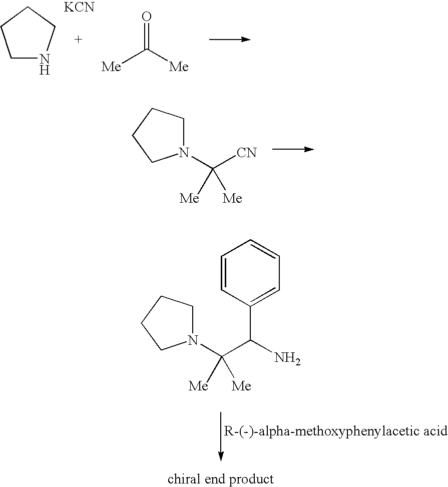

The present invention also provides a new and convenient route to chiral 1,2-diamines, which does not involve the use of cyanide or phenyllithium.

Thus, in a second aspect, the invention also provides a process for the formation of a compound of formula (I):

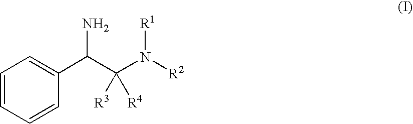

wherein:
R¹ and R² are independently selected from hydrogen and C₁₋₄alkyl, optionally substituted with one or more groups Y; or R¹ and R² together with the nitrogen atom to which they are attached form a saturated or partially unsaturated 4-, 5-, 6- or 7-membered carbocyclic ring optionally substituted with a group Y′;
Y is selected from the group consisting of C₁₋₄alkoxy, hydroxy, haloC₁₋₄alkoxy and C₃₋₅cycloalkyl;
Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH₂— or —CH₂—CH₂— bridge between two atoms on the 4-, 5-, 6-, or 7-membered carbocyclic ring;
R³ and R⁴ are independently C₁₋₄alkyl, optionally substituted with one or more groups X; or R³ and R⁴ together with the carbon atom to which they are attached form a saturated 5- or 6-membered carbocyclic ring optionally substituted with one or more groups X′, in the case of R³ and R⁴ together with the carbon atom to which they are attached forming a 5-membered saturated carbocyclic ring, that ring may optionally further comprise an additional heteroatom group selected from O, N and S(O)ₘ; where m=0, 1 or 2;
X is selected from the group consisting of halogen, hydroxy, C₁₋₄alkoxy, haloC₁₋₄alkyl, haloC₁₋₄alkoxy and C₅₋₁₀aryl; and
X′ is selected from the group consisting of halogen, hydroxy, C₁₋₄alkyl, C₁₋₄alkoxy, haloC₁₋₄alkyl, haloC₁₋₄alkoxy and C₅₋₁₀aryl;
whereby R¹, R², R³ and R⁴ are not all simultaneously unsubstituted methyl;

the process comprising reducing a compound of formula (II):

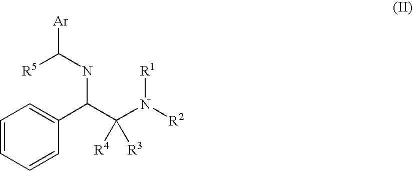

wherein R¹, R², R³ and R⁴ are as defined for formula (I), R⁵ is C₁₋₄alkyl and Ar is optionally substituted phenyl; using hydrogen and a palladium catalyst.

In one embodiment, the reaction takes place at an elevated temperature.

In one embodiment, the reaction takes place in an alcoholic solvent. In one embodiment, the solvent is ethanol or methanol. In one embodiment, the solvent is methanol. In one embodiment, the reaction takes place in ethyl acetate.

In one embodiment, in order to provide a faster reaction time, the reaction takes place in the presence of an organic acid or sulphuric acid. In one embodiment, the acid is sulphuric acid. In one embodiment, the acid is an organic acid, such as acetic acid or formic acid.

In one embodiment, the palladium catalyst is 10% palladium on charcoal (10% Pd/C).

In one embodiment, the reaction comprises treatment of (II) with hydrogen gas and 10% palladium on charcoal (10% Pd/C) in an alcoholic solvent in the presence of an organic acid or sulphuric acid.

In one embodiment, the reaction comprises treatment of (II) with hydrogen gas and 10% palladium on charcoal (10% Pd/C) in methanol in the presence of sulphuric acid.

In one embodiment, the reaction comprises treatment of (II) with formic acid and 10% palladium on charcoal (10% Pd/C) (CTH reduction) followed by hydrolysis under acidic conditions.

In one embodiment, R¹ and R² together with the nitrogen atom to which they are attached form a saturated 4-, 5- 6-or 7-membered carbocyclic ring.

In one embodiment, R³ and R⁴ are independently C₁₋₄alkyl. In one embodiment, R³ and R⁴ are both methyl.

In formula (II), Ar is optionally substituted phenyl. The number and type of substituents on the phenyl ring is not critical, although very strong electron withdrawing groups may have an effect on the enantiomeric selectivity of the reaction. In one embodiment, Ar is phenyl optionally substituted by one, two or three substituents selected from the group consisting of C₁₋₄alkyl, C₁₋₄alkoxy, halo, haloC₁₋₄alkyl, haloC₁₋₄alkoxy, C₁₋₄alkylthio, C₃₋₆cycloalkyl, C₁₋₄alkoxyC₁₋₄alkyl and cyano.

In one embodiment, Ar is unsubstituted phenyl.

In one embodiment, R⁵ is methyl.

In one embodiment, the process provides a compound of formula (I) in the R configuration. In another embodiment, the process provides a compound of formula (I) in the S configuration.

In one embodiment, the process gives a compound of formula (I) with at least 90% enantiomeric excess. In one embodiment, the process gives a compound of formula (I) with at least 95% enantiomeric excess. In one embodiment, the process gives a compound of formula (I) with at least 99% enantiomeric excess.

In one embodiment, the process gives a compound of formula (Ia):

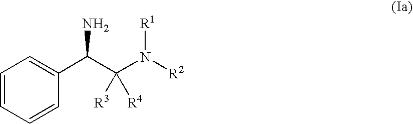

wherein R¹, R², R³ and R⁴ are as defined for formula (I), in at least 90% enantiomeric excess. In one embodiment, the process gives a compound of formula (Ia) in at least 95% enantiomeric excess. In one embodiment, the process gives a compound of formula (Ia) in at least 99% enantiomeric excess.

In one embodiment, the invention provides a process for the formation of 2-methyl-1-phenyl-2-(1-pyrrolidinyl)propyl]amine, the process comprising reducing a compound of formula (IIa):

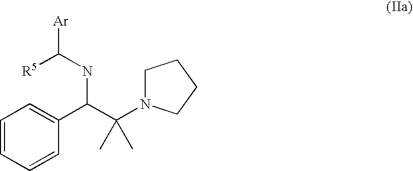

wherein R⁵ is C₁₋₄alkyl and Ar is optionally substituted phenyl; using hydrogen and a palladium catalyst.

In one embodiment, the process provides [(1R)-2-methyl-1-phenyl-2-(1-pyrrolidinyl)propyl]amine. In one embodiment, the process provides [(1S)-2-methyl-1-phenyl-2-(1-pyrrolidinyl)propyl]amine.

In one embodiment, the the invention provides a process for the formation of 2-methyl-1-phenyl-2-(1-pyrrolidinyl)propyl]amine, the process comprising reducing N-[2-methyl-1-phenyl-2-(1-pyrrolidinyl)propyl]-α-methylbenzylamine, using hydrogen and a palladium catalyst.

In one embodiment, the present invention provides a process for the formation of [(1R)-2-methyl-1-phenyl-2-(1-pyrrolidinyl)propyl]amine, the process comprising reducing [(1R)-2-methyl-1-phenyl-2-(1-pyrrolidinyl)propyl]-R-(+)-α-methylbenzylamine or [(1R)-2-methyl-1-phenyl-2-(1-pyrrolidinyl)propyl]-S-(−)-α-methylbenzylamine, using hydrogen and a palladium catalyst.

[(1R)-2-methyl-1-phenyl-2-(1-pyrrolidinyl)propyl]-R-(+)-α-methylbenzylamine and [(1R)-2-methyl-1-phenyl-2-(1-pyrrolidinyl)propyl]-S-(−)-α-methylbenzylamine are shown below:

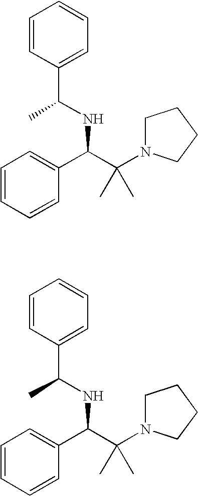

In one embodiment, the process provides [(1R)-2-methyl-1-phenyl-2-(1-pyrrolidinyl)propyl]amine in at least 90% enantiomeric excess. In one embodiment, the process provides [(1R)-2-methyl-1-phenyl-2-(1-pyrrolidinyl)propyl]amine in at least 95% enantiomeric excess. In one embodiment, the process provides [(1R)-2-methyl-1-phenyl-2-(1-pyrrolidinyl)propyl]amine in at least 99% enantiomeric excess.

In another aspect, the present invention provides [(1R)-2-methyl-1-phenyl-2-(1-pyrrolidinyl)propyl]-R-(+)-α-methylbenzylamine or a salt or solvate thereof:

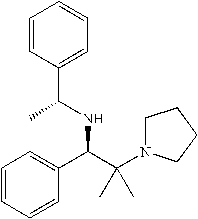

The present invention also provides a process for the formation of a compound of formula (II) as defined above, comprising:

(i) reaction of a compound of formula (VIII):

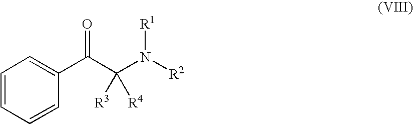

wherein R¹, R², R³ and R⁴ are as defined for formula (I), with a compound of formula (IV):

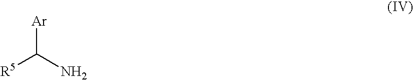

wherein R⁵ is C₁₋₄alkyl and Ar is phenyl optionally substituted by one or more groups; followed by

(ii) reduction with a sodium borohydride derivative.

It is believed that the reaction between a compound of formula (VIII) and a compound of formula (IV) result in the following imine intermediate:

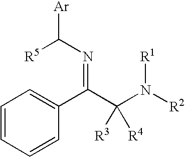

which is then reduced in step (ii) to a compound of formula (II).

In one embodiment, treatment of alpha-aminoketone (VIII) with chiral amine (IV) is carried out in an aprotic solvent in the presence of titanium (IV) chloride and a tertiary base. In a further embodiment, the aprotic solvent is acetonitrile or methylene chloride. In a further embodiment the aprotic solvent is acetonitrile.

In one embodiment, treatment of alpha-aminoketone (VIII) with chiral amine (IV) is carried out at elevated temperature.

In one embodiment, treatment of alpha-aminoketone (VIII) with chiral amine (IV) is carried out in toluene in the presence of a strong acid catalyst and water is removed from the reaction mixture by distillation. In an alternative embodiment, treatment of alpha-aminoketone (VIII) with chiral amine (IV) is carried out in toluene in the presence of a drying agent.

In one embodiment, the reduction step (ii) is achieved using a reducing agent selected from a sodium borohydride derivative, lithium borohydride and lithium aluminum hydride, in a solvent selected from C₁₋₄alcohol. In a further embodiment, the reducing agent is selected from sodium borohydride, sodium triacetoxyborohydride, sodium cyanoborohydride, lithium borohydride and lithium aluminum hydride. In one embodiment, the reducing agent is sodium triacetoxyborohydride or sodium cyanoborohydride. In one embodiment, the reducing agent is sodium borohydride. In one embodiment, the solvent is methanol.

In one embodiment, R¹ and R² together with the nitrogen atom to which they are attached form a saturated 4-, 5- 6- or 7-membered carbocyclic ring.

In one embodiment, R³ and R⁴ are independently C₁₋₄alkyl. In one embodiment, R³ and R⁴ are both methyl.

In one embodiment, Ar is phenyl.

In one embodiment, R⁵ is methyl.

In one embodiment, the compound of formula (IV) is α-methylbenzylamine. In one embodiment, the compound of formula (IV) is R-(+)-α-methylbenzylamine. In one embodiment, the compound of formula (IV) is S-(−)-α-methylbenzylamine.

In one embodiment, the present invention provides a process for the formation of a compound of formula (IIa) as defined above, comprising:

(i) reaction of 2-pyrrolidinyl-2-methylpropiophenone:

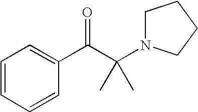

with a compound of formula (IV):

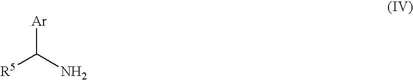

wherein R⁵ is C₁₋₄alkyl and Ar is optionally substituted phenyl; followed by

(ii) reduction with a sodium borohydride derivative.

In one embodiment, the compound of formula (IV) is R-(+)-α-methylbenzylamine and the compound of formula (II) obtained is [(1R)-2-methyl-1-phenyl-2-(1-pyrrolidinyl)propyl]-R-(+)-α-methylbenzylamine]:

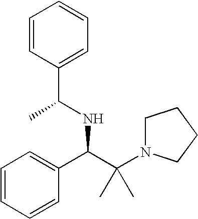

In one embodiment, the present invention also provides a process for the formation of [(1R)-2-methyl-1-phenyl-2-(1-pyrrolidinyl)propyl] R-(+)-α-methylbenzylamine, comprising reaction of 2-pyrrolidinyl-2-methylpropiophenone with R-(+)-α-methylbenzylamine, followed by reduction with a sodiumborohydride derivative.

In a further aspect, the present invention provides a process for the formation of a compound of formula (VIII), comprising treatment of a compound of formula (V):

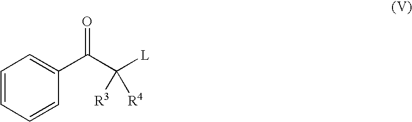

wherein R³ and R⁴ are as defined for formula (I) and L is a leaving group, with a compound of formula (VI):

R⁶—OH (VI)

wherein R⁶ is C₁₋₄alkyl, in the presence of a base, followed by reaction with a compound of formula (VII):

NHR¹R² (VII)

wherein R¹ and R² are as defined for formula (I).

In one embodiment, in formula (V), R¹ and R² together with the nitrogen atom to which they are attached form a saturated 4-, 5- 6-or 7-membered carbocyclic ring. In one embodiment, R¹ and R² together with the nitrogen atom to which they are attached form a pyrrolidine.

In one embodiment, in formula (V), R³ and R⁴ are independently C₁₋₄alkyl. In one embodiment, R³ and R⁴ are both methyl.

In one embodiment, L is halogen. In one embodiment, L is bromine.

In one embodiment, the compound of formula (VI) is ethanol or methanol. In one embodiment, the compound of formula (VI) is methanol.

In one embodiment, the base is selected from the group consisting of carbonates, hydrogen carbonates, inorganic amides, hydrides or inorganic alkoxides. In one embodiment the base is selected from potassium carbonate, sodium carbonate, potassium hydrogen carbonate, sodium hydride, NaOR⁷ (wherein R⁷ is C₁₋₄alkyl) or sodium hydride. In one embodiment the base is potassium carbonate.

It is believed that treatment of a compound of formula (V) with a compound of formula (VI) gives an epoxy compound:

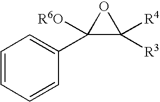

In one embodiment, the present invention provides a process for the formation of 2-pyrrolidinyl-2-methylpropiophenone, comprising treatment of a compound of formula (Va):

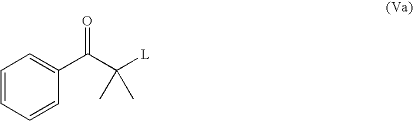

wherein L is a leaving group, with a compound of formula (VI):

R⁶—OH (VI)

wherein R⁶ is C₁₋₄alkyl, in the presence of a base, followed by reaction with pyrrolidine.

In one embodiment, the present invention provides a process for the formation of 2-pyrrolidinyl-2-methylpropiophenone, comprising treating 2-bromoisobutyrophenone with ethanol or methanol, in the presence of a base, followed by reacting with pyrrolidine.

In one embodiment, the present invention provides a process for the formation of 2-pyrrolidinyl-2-methylpropiophenone, comprising treating 2-bromoisobutyrophenone with methanol, in the presence of a base, followed by reacting with pyrrolidine.

In one embodiment, the present invention provides a process for the formation of 2-pyrrolidinyl-2-methylpropiophenone, comprising treating 2-bromoisobutyrophenone with ethanol or methanol, in the presence of potassium carbonate, followed by reacting with pyrrolidine.

In one embodiment, the present invention provides a process for the formation of 2-pyrrolidinyl-2-methylpropiophenone, comprising treating 2-bromoisobutyrophenone with methanol, in the presence of potassium carbonate, followed by reacting with pyrrolidine.

In one embodiment, the present invention provides a process for the formation of 2-(methyloxy)-N-[2-methyl-1-phenyl-2-(1-pyrrolidinyl)propyl]-4,6-bis(trifluoromethyl)benzamide comprising:

i) the formation of 2-pyrrolidinyl-2-methylpropiophenone, by treatment of a compound of formula (Va):

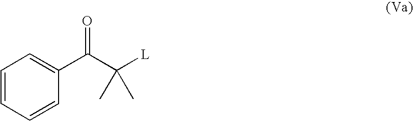

wherein L is a leaving group, with a compound of formula (VI):

R⁶—OH (VI)

wherein R⁶ is C₁₋₄alkyl, in the presence of a base, followed by reaction with pyrrolidine;

ii) reaction of 2-pyrrolidinyl -2-methylpropiophenone with a compound of formula (IV):

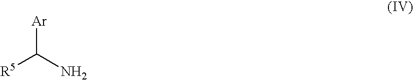

wherein R⁵ is C₁₋₄alkyl and Ar is optionally substituted phenyl; followed by reduction with a sodium borohydride derivative;

iii) reduction of the product of step ii) using hydrogen and a palladium catalyst; and

iv) reaction of the product of step iii) or a salt thereof with a compound selected from the group consisting of [2-(methyloxy)-4,6-bis(trifluoromethyl)phenyl]carbonyl C₁₋₆alkyl sulfone, [2-(methyloxy)-4,6-bis(trifluoromethyl)phenyl]carbonyl aryl sulfone and diC₁₋₆alkyl [2-(methyloxy)-4,6-bis(trifluoromethyl)phenyl]carbonyl phosphate.

In one embodiment, the present invention provides a process for the formation of 2-(methyloxy)-N-[2-methyl-1-phenyl-2-(1-pyrrolidinyl)propyl]-4,6-bis(trifluoromethyl)benzamide comprising:

i) the formation of 2-pyrrolidinyl-2-methylpropiophenone, by treatment of 2-bromoisobutyrophenone with methanol, in the presence of potassium carbonate, followed by reaction with pyrrolidine;

ii) reaction of 2-pyrrolidinyl -2-methylpropiophenone with R-(+)-α-methylbenzylamine; followed by reduction with sodium borohydride;

iii) reduction of the product of step ii) using hydrogen and a palladium catalyst; and

iv) reaction of the product of step iii) or a salt thereof with [2-(methyloxy)-4,6-bis(trifluoromethyl)phenyl]carbonyl propyl sulfone.

In a third aspect, the present invention provides 2-pyrrolidinyl-2-methylpropiophenone or a salt or solvate thereof:

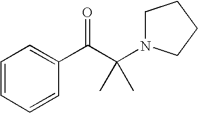

The invention is further illustrated by the following non-limiting examples.

### ABBREVIATIONS

AcCN Acetonitrile

TEA Triethylaimine

MTBE Methyl t-butyl ether

NMR Nuclear magnetic resonance

EtOAc Ethyl acetate

LCMS Liquid chromatography mass spectrometry

RT Retention time

SP Sharp peak

RP Round peak

Conc. Concentrated

HPLC High performance liquid chromatography

### EXAMPLE 1

### Synthesis of 2-pyrrolidinyl -2-methylpropiophenone

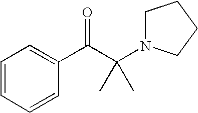

In methanol:

2-Bromoisobutyrophenone (0.1 mol, 22.7 g) and K₂CO₃ 99% (46 g, 3.3 eq) in methanol (HPLC grade, 50 mL,) were stirred at room temperature for 3 hours under nitrogen atmosphere. When the ¹H-NMR spectrum showed complete reaction, the reaction mixture was filtered and the solid washed with MTBE (100 ml). The organic layer was concentrated to about 50 ml then pyrrolidine 99.5% (15 mL) was added to the slurry and heated at 75-85° C. for 18 hours. The reaction mixture was reduced by evaporation to about 30 ml, then EtOAc (25 ml) was added and the organic solution extracted with a 4N HCl solution (2×25 ml). The combined aqueous solutions were basified with a 15% NaOH solution (˜35 ml) and extracted with EtOAc (50 ml). The aqueous solution was extracted again with EtOAc (2×25 ml) and the combined organic solutions were evaporated to give the desired compound (17.27 g) (assay 95% a/a, yield ˜80%).

In ethanol:

2-Bromoisobutyrophenone (0.015 mol, 3.37 g, 2.5 mL) and K₂CO₃ 99% (6 g, 1.8 eq) in ethanol (9 ml, 2.7 eq/vol) were stirred at room temperature for 20 hours under nitrogen atmosphere. Pyrrolidine 99.5% (3.5 mL, 1 eq/vol) was added to the slurry and heated at 70° C. for 24 hours. The solution was diluted with EtOAc (20 mL) and filtered. The residue was washed with EtOAc (20 ml) and the combined organic layer was washed with water (20 mL) and extracted with 2 N HCl (5 mL×2). The acid solution was washed with EtOAc (20 mL) then EtOAc (20 mL) was added followed by a saturated solution of K₂CO₃ (20 ml). The EtOAc layer was separated and the aqueous layer re-extracted with EtOAc (20 ml). The combined organic solutions were washed with water (2×20 mL) and concentrated to give the desired product (2.62 g, 81% yield).

### EXAMPLE 2

### Synthesis of [(1R)-2-methyl-1-phenyl-2-(1-pyrrolidinyl)propyl]-R-(+)-α-methylbenzylamine]

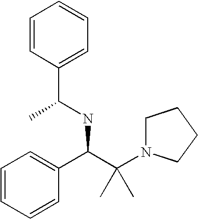

Method 1:

A flask was charged with (±)-2-methyl-1-phenyl-2-(1-pyrrolidinyl)propan-1-one (3.9 g, 18 mmol, 1 wt), triethylamine 99.5% (10 mL, 2.5 vol) and R-(+)-α-methylbenzylamine (2.8 mL, 0.7 vol) in acetonitrile (35 mL, 9 vol) under nitrogen atmosphere. The solution was kept at 10° C. (water-ice bath) and 1 M titanium(IV) chloride in dichloromethane (14.3 mL, 3.7 vol) was added dropwise in 15 min with vigorous stirring. The resulting slurry was kept at room temperature for 2.5 hours (HPLC after mini workup: a reaction sample was diluted in methanol, sodium borohydride was added and quenched with 2 N hydrochloric acid; RT_{RP} 4.28, RT_{SP} 2.49; 8 min method; 95% a/a). The mixture was cooled to 0° C. and sodium borohydride (1.40 g, 0.36 wt) was added portionwise followed by dropwise addition of methanol (8 mL). The reaction was slowly brought to room temperature in 2 hours and left overnight with stirring (LCMS, RT_{SP} 4.3, RT_{SP}, no resolution; MH⁺_{SP} 321, MH⁺_{RP} 323). The slurry was quenched with water (4 mL, 1 vol), filtered and the filter was rinsed with EtOAc (8 mL, 2 vol×2). The solvent was partially evaporated to about ˜10 vol. The solution diluted with EtOAc (20 ml) was extracted with 2 N HCl (20 ml) and separated. The EtOAc was extracted again with 2N HCl (8 ml) then the combined aqueous layer was brought to pH 12 with 6N NaOH and extracted with EtOAc (8 ml×3). Evaporation of the solvent gave the title compound as a yellow-dark oil (5.4 g, 100% yield, 94% a/a).

Method 2:

A flask was charged with (±)-2-methyl-1-phenyl-2-(1-pyrrolidinyl)propan-1-one (1 g, 4.6 mmol, 1 wt), triethylamine 99.5% (2.5 mL, 2.5 vol) and R-(+)-α-methylbenzylamine (0.7 mL, 0.7 vol) in acetonitrile (8mL, 9vol) under nitrogen atmosphere at 15-20° C. A solution of 1 M titanium(IV) chloride in dichloromethane (4.6 ml, 1 eq) was added dropwise in 15 min at 10° C. (water-ice bath) with vigorous stirring. The funnel was washed with acetonitrile (2 ml). The resulting slurry was kept at room temperature for 1.5 hours. Sodium borohydride (350 mg, 0.36 wt) was added followed by a dropwise addition (20 min) of methanol (4 mL). The reaction was stirred at 20° C. for 2 hours. The reaction was not complete therefore methanol (2 ml) was added and the reaction stirred again for 1 hour The solvent was evaporated under vacuum to 5 vol and EtOAc (10 mL) was added. The suspension was filtered on Sterimat and the solid washed with EtOAc (5 mL). The EtOAc layer was extracted with 2N HCl solution (10 ml×2) and the combined aqueous solution was washed with EtOAc (10 mL). EtOAc (10 mL) was added and the combined aqueous layer was brought to pH 12 with solid KOH. The EtOAc was separated and the aqueous phase re-extracted with EtOAc (10 mL). The organic phases were combined, washed with water (2×10 mL) and evaporated to give the desired compound (1.45 g, 97%).

### EXAMPLE 3

### Synthesis of [(1R)-2-methyl-1-phenyl-2-(1-pyrrolidinyl)propyl]amine

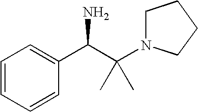

Method 1 (H₂SO₄, Single Step):

An endeavor tube was charged [(1R)-2-methyl-1-phenyl-2-(1-pyrrolidinyl)propyl]-R-(+)-α-methylbenzylamine] (1.62 g, 1 wt) dissolved in 10% conc. sulphuric acid in methanol (HPLC grade, 3 mL, 2 vol). To the solution was added 10% palladium/carbon (150 mg, 10% wt; Strem Chemicals, 50% wet) and submitted for 1 hour at 60° C. and 3 atm of hydrogen. The mixture was left to reach room temperature and filtered over Celite®. The filter was rinsed with methanol (4 mL×2, 5 vol) and evaporated to 5 volumes. The pale yellow solution was added to 1 N HCl (6 mL, 4 vol), extracted with EtOAc (6 mL, 4 vol) and separated. The organic phase was extracted with 2 N HCl (2 vol) and the combined water layer was basified to pH 12-13 with 6 N NaOH (4 mL, 3 vol). The resulting milky solution gave a white solid after stirring at room temperature at 10° C. for 1 hour. The solid was washed with water (1 vol). After filtration and drying in the oven at 30° C. overnight the title compound was recovered as a white solid (890 mg, 76%).

Method 2 (CH₃COOH, Two Steps):

An endeavor tube was charged with [(1R)-2-methyl-1-phenyl-2-(1-pyrrolidinyl)propyl]-R-(+)-α-methylbenzylamine] (1.0 g, 1 wt) dissolved in glacial acetic acid (2 mL, 2 vol) and methanol (2 mL, 2 vol). 10% palladium/carbon (100 mg, 0.1 wt; Strem Chemicals, 50% wet) was added to the solution and submitted for 2 hours at 5 atm of hydrogen. The reaction was slow, therefore the hydrogenation was continued for 12 hours at 50° C. and 5 atm of hydrogen (the reaction was always stopped at room temperature during the night). The mixture was left to reach room temperature and filtered over Celite®. The filter was rinsed with methanol (10 mL, 10 vol) and evaporated to 2 volumes. The pale yellow solution was diluted with 4 N HCl (2 mL, 2 vol) and heated at 100° C. for 6 h. The mixture was brought at room temperature, water was added (8 mL) and extracted with EtOAc (4 vol×2). The aqueous layer was basified to pH 12-13 with 15% NaOH (about 5 mL, 5 vol) and left at 30° C. for 1 h. The resulting solid was filtered, washed with water (2 ml) and dried to give the desired compound (460 mg, 68%).

Method 3 (CTH, 2 Steps):

[(1R)-2-methyl-1-phenyl-2-(1-pyrrolidinyl)propyl]-R-(+)-α-methylbenzylamine] (1.45 g, 1 wt) was dissolved in formic acid (5 mL, 3.4 vol) and EtOAc (0.5 mL). 10% Palladium/carbon (300 mg, 10% wt; Strem Chemicals, 50% wet) was added and the suspension heated to 100° C. After stirring for 2 hours the reaction was complete, giving the formyl derivative. The suspension was cooled to 40-50° C. and filtered on Sterimat. The Pd/C was washed with EtOAc (5 mL×2). The solvent was evaporated to 3-4 total vol then a solution of 4 N HCl in water (10 mL) was added. The solution was stirred at 100° C. for 3 hours, then at room temperature the aqueous layer was washed with EtOAc (10 mL×2). EtOH (2 mL) was added and the solution cooled at 10° C. then NaOH 30% was added to pH 12-13. A solid was obtained and after 1 hour filtered and washed with cold water (5 ml×2). The solid was dried for 14 hours under vacuum at 25° C. giving the desired compound (710 mg, 71%).

### EXAMPLE 4

### 2-(methyloxy)-N-[2-methyl-1-phenyl-2-(1-pyrrolidinyl)propyl]-4,6-bis(trifluoromethyl)benzamide

### Example 4.1

### Mesylchloride Method

2,4-Ditrifluoromethyl-6-methoxy-benzoic acid (150 mg, 0.55 mmol) was suspended in AcCN (1.5 ml, 10 vol), TEA (0.1 ml, 1.4 eq) was added and the mixture cooled to 0° C. Mesylchloride (0.054 ml, 0.7 mmol) was added and the mixture stirred for 30 min.

[2-Methyl-1-phenyl-2-(1-pyrrolidinyl)propyl]amine (100 mg, 0.5 mmol) was added and after stirring for 15 min, TEA (0.1 ml, 1.4 eq) was added and the obtained suspension stirred for 10 min.

Methanol (0.2 ml) was added and after stirring for 30 min the solvent was partially evaporated and the obtained suspension was diluted with EtOAc (2 ml) washed with water, a 1% bicarbonate solution , water and evaporated to give the desired 2-(methyloxy)-N-[2-methyl-1-phenyl-2-(1-pyrrolidinyl)propyl]-4,6-bis(trifluoromethyl)benzamide (200 mg, ˜80% yield).

### Example 4.2

### Tosylchloride Method

2,4-Ditrifluoromethyl-6-methoxy-benzoic acid (300 mg, 1.1 mmol) was suspended in AcCN (3 ml, 10 vol), TEA (0.2 ml, 1.4 eq) was added and the mixture cooled to 0° C. Tosyl chloride (200 mg, 0.7 mmol) was added and the mixture stirred for 30 min, then [2-methyl-1-phenyl-2-(1-pyrrolidinyl)propyl]amine (200 mg, 1 mmol) was added. After stirring for 15 min, TEA (0.2 ml, 1.4 eq) was added and the obtained suspension stirred for 30 min at 0° C.

The solvent was partially evaporated and the obtained suspension was diluted with EtOAc (5 ml), washed with water (5 ml), 1M NaOH in water (2×5 ml), water (5 ml) and evaporated to give the desired 2-(methyloxy)-N-[2-methyl-1-phenyl-2-(1-pyrrolidinyl)propyl]-4,6-bis(trifluoromethyl)benzamide (500 mg, 90% a/a purity, ˜70% yield).

### Example 4.3

### Diethylchlorophosphate Method

2,4-Ditrifluoromethyl-6-methoxy-benzoic acid (1.58 g, 5.5 mmol) was suspended in AcCN (15 ml, 10 vol), TEA (1.4 ml, 10 mmol) was added and the mixture cooled to 0° C. Diethylchlorophosphate (0.8 ml, 5.5 mmol) was added in 5 min and the mixture stirred for 1 hr 30 min.

In a different flask the chiral salt of [2-methyl-1-phenyl-2-(1-pyrrolidinyl)propyl]amine (1.92 gr, 5 mmol) was dissolved in CH₂Cl₂ (20 ml, 10 vol) and treated with 1M NaOH, the organic phase was separated, the water was re-extracted with CH₂Cl₂ (10 ml) and the combined organic layer was evaporated to ˜10 ml total volume. The obtained CH₂Cl₂ solution was added over 15 min to the activated acid solution cooled to ˜5° C.

At the end of the addition the reaction was complete and after 10 min the solvent was partially evaporated to 10 ml, EtOAc (20 ml) was added, washed with 1M NaOH (2×15 ml), water (2×15 ml) and then evaporated to give the desired 2-(methyloxy)-N-[2-methyl-1-phenyl-2-(1-pyrrolidinyl)propyl]-4,6-bis(trifluoromethyl)benzamide (2.33 gr, 94% yield)

### Example 4.4

### Mesylchloride Method Using Amine Salt

2,4-ditrifluoromethyl-6-methoxy-benzoic acid (1.58 g, 5.5 mmol) was suspended in AcCN (15 ml, 10 vol), TEA (0.83 ml, ˜1.4 eq) was added and the mixture cooled to −10° C. After 5 min mesylchloride (0.42 ml) was added and the mixture stirred for 30 min. CH₂Cl₂ (15 ml) was added and the mixture cooled to −15° C. The chiral salt of [2-methyl-1-phenyl-2-(1-pyrrolidinyl)propyl]amine (2 gr, 5.2 mmol) was added followed by CH₂Cl₂ (5 ml) and TEA (0.8 ml).

The reaction mixture was stirred at −15° C. for 1 hour then water (2 ml) was added and the reaction mixture partially evaporated to about 5 vol.

The obtained suspension was diluted with EtOAc (20 ml) washed with 1M NaOH (2×20 ml), water (2×20 ml) and evaporated to give the desired 2-(methyloxy)-N-[2-methyl-1-phenyl-2-(1-pyrrolidinyl)propyl]-4,6-bis(trifluoromethyl)benzamide (2.4 g, 94% yield).

### Example 4.5

### Tosylchloride Method Using Amine Salt

2,4-Ditrifluoromethyl-6-methoxy-benzoic acid (1.7 g, 6 mmol) was suspended in AcCN (17 ml, 10 vol) and cooled to −10° C. TEA (0.77 ml, ˜5 mmol) was added followed by tosylchloride (1 gr). The mixture was stirred for 20 min then TEA (1.6 ml) was added followed by the chiral salt of [2-methyl-1-phenyl-2-(1-pyrrolidinyl)propyl]amine (1.9 gr, 5 mmol) and CH₂Cl₂ (10 ml). The reaction temperature increased to 0° C. and the mixture was stirred at this temperature for 30 min.

The solvent was partially evaporated and the obtained suspension was diluted with EtOAc (20 ml) washed with 1M NaOH (2×20 ml), water (2×20 ml) and evaporated to give the desired 2-(methyloxy)-N-[2-methyl-1-phenyl-2-(1-pyrrolidinyl)propyl]-4,6-bis(trifluoromethyl)benzamide (2 g, 80% a/a purity, ˜70% yield).

### Example 4.6

### Diethylchlorophosphate Method Using Amine Salt

2,4-Ditrifluoromethyl-6-methoxy-benzoic acid (1.58 g, 5.5 mmol) was suspended in AcCN (15 ml, 10vol), TEA ((1.4 ml, 10 mmol) was added and the mixture cooled to 0° C. Diethylchlorophosphate (0.8 ml, 5.5 mmol) was added in 5 min and the mixture stirred for 1 hr 30 min.

The mixture was cooled to −20° C., CH₂Cl₂ (10 ml) was added followed by the chiral salt of [2-methyl-1-phenyl-2-(1-pyrrolidinyl)propyl]amine (1.92 gr, 5 mmol) and CH₂Cl₂ (5 ml). After stirring for 5 min TEA (0.8 ml) was added and the obtained suspension was stirred at −10° C. for 1 hr 30 min.

The solvent was partially evaporated (˜10 vol) and EtOAc (20 ml) was added, washed with 1M NaOH (2×15 ml), water (2×15 ml) and then evaporated to give the desired 2-(methyloxy)-N-[2-methyl-1-phenyl-2-(1-pyrrolidinyl)propyl]-4,6-bis(trifluoromethyl)benzamide (2.3 gr, 96% a/a purity, ˜90% yield).

### EXAMPLE 5

### Synthesis of [(1R)-2-methyl-1-phenyl-2-(1-pyrrolidinyl)propyl]amine

### Example 5.1

### Synthesis of 2-pyrrolidinyl -2-methylpropiophenone

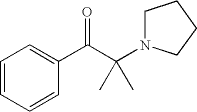

2-Bromoisobutyrophenone (0.1 mol, 22.7 g) and K₂CO₃ 99% (46 g, 3.3 eq) in methanol (HPLC grade, 50 mL,) were stirred at room temperature for 3 hours under nitrogen atmosphere. When the ¹H-NMR spectrum showed complete reaction, the reaction mixture was filtered and the solid washed with MTBE (100 ml). The organic layer was concentrated to about 50 ml then pyrrolidine 99.5% (15 mL) was added to the slurry and heated at 75-85° C. for 18 hours. The reaction mixture was reduced by evaporation to about 30 ml, then EtOAc (25 ml) was added and the organic solution extracted with a 4N HCl solution (2×25 ml). The combined aqueous solutions were basified with a 15% NaOH solution (˜35 ml) and extracted with EtOAc (50 ml). The aqueous solution was extracted again with EtOAc (2×25 ml) and the combined organic solutions were evaporated to give the desired compound (17.27 g) (assay 95% a/a, yield ˜80%).

### Example 5.2

### Synthesis of [(1R)-2-methyl-1-phenyl-2-(1-pyrrolidinyl)propyl]-R-(+)-α-methylbenzylamine]

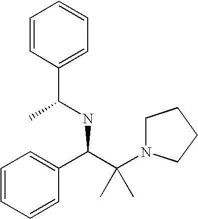

A flask was charged with (±)-2-methyl-1-phenyl-2-(1-pyrrolidinyl)propan-1-one (3.9 g, 18 mmol, 1 wt), triethylamine 99.5% (10 mL, 2.5 vol) and R-(+)-α-methylbenzylamine (2.8 mL, 0.7 vol) in acetonitrile (35 mL, 9 vol) under nitrogen atmosphere. The solution was kept at 10° C. (water-ice bath) and 1 M titanium(IV) chloride in dichloromethane (14.3 mL, 3.7 vol) was added dropwise in 15 min with vigorous stirring. The resulting slurry was kept at room temperature for 2.5 hours (HPLC after mini workup: a reaction sample was diluted in methanol, sodium borohydride was added and quenched with 2 N hydrochloric acid; RT_{RP} 4.28, RT_{SP} 2.49; 8 min method; 95% a/a). The mixture was cooled to 0° C. and sodium borohydride (1.40 g, 0.36 wt) was added portionwise followed by dropwise addition of methanol (8 mL). The reaction was slowly brought to room temperature in 2 hours and left overnight with stirring (LCMS, RT_{SP} 4.3, RT_{SP}, no resolution; MH⁺_{SP} 321, MH⁺_{RP} 323). The slurry was quenched with water (4 mL, 1 vol), filtered and the filter was rinsed with

EtOAc (8 mL, 2 vol×2). The solvent was partially evaporated to about ˜10 vol. The solution diluted with EtOAc (20 ml) was extracted with 2 N HCl (20 ml) and separated. The EtOAc was extracted again with 2N HCl (8 ml) then the combined aqueous layer was brought to pH 12 with 6N NaOH and extracted with EtOAc (8 ml×3). Evaporation of the solvent gave the title compound as a yellow-dark oil (5.4 g, 100% yield, 94% a/a).

### Example 5.3

### Synthesis of [(1R)-2-methyl-1-phenyl-2-(1-pyrrolidinyl)propyl]amine

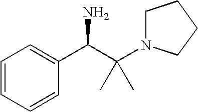

An endeavor tube was charged [(1R)-2-methyl-1-phenyl-2-(1-pyrrolidinyl)propyl]-R-(+)-α-methylbenzylamine] (1.62 g, 1 wt) dissolved in 10% conc. sulphuric acid in methanol (HPLC grade, 3 mL, 2 vol). To the solution was added 10% palladium/carbon (150 mg, 10% wt; Strem Chemicals, 50% wet) and submitted for 1 hour at 60° C. and 3 atm of hydrogen. The mixture was left to reach room temperature and filtered over Celite®. The filter was rinsed with methanol (4 mL×2, 5 vol) and evaporated to 5 volumes. The pale yellow solution was added to 1 N HCl (6 mL, 4 vol), extracted with EtOAc (6 mL, 4 vol) and separated. The organic phase was extracted with 2 N HCl (2 vol) and the combined water layer was basified to pH 12-13 with 6 N NaOH (4 mL, 3 vol). The resulting milky solution gave a white solid after stirring at room temperature at 10° C. for 1 hour. The solid was washed with water (1 vol). After filtration and drying in the oven at 30° C. overnight the title compound was recovered as a white solid (890 mg, 76%).

### EXAMPLE 6

### 2-(methyloxy)-N-[2-methyl-1-phenyl-2-(1-pyrrolidinyl)propyl]-4,6-bis(trifluoromethyl)benzamide

(2R)-(methyloxy)(phenyl)ethanoic acid—[(1R)-2-methyl-1-phenyl-2-(1-pyrrolidinyl)propyl]amine (1:1) (3.05 g) was suspended in ethyl acetate (30 ml) and an aqueous solution of ammonium hydroxide 14% (15.25 ml) was added. The reaction mixture was stirred until complete dissolution and the phases were separated. The organic phase was washed with water (15 ml.) and concentrated to 7.6 ml giving a solution of the chiral intermediate [(1R)-2-methyl-1-phenyl-2-(1-pyrrolidinyl)propyl]amine free base.

2-(methyloxy)-4,6-bis(trifluoromethyl)benzoic acid (2.51 g) was dissolved at 20° C. in ethyl acetate (23 ml) and triethylamine (2.21 ml) was added. The reaction mixture was cooled at 0° C. and 1-propanesulfonyl chloride (0.98 ml.) was added keeping the internal temperature under 5° C. The reaction mixture was stirred for 1 hr at 0° C. The solution of the free base of [(1R)-2-methyl-1-phenyl-2-(1-pyrrolidinyl)propyl]amine was added at 0° C. keeping the internal temperature under 10° C. The reaction mixture was stirred overnight at room temperature. The reaction mixture was quenched with water (15 ml) and the phases were separated. The organic phase was washed twice with an aqueous solution of sodium hydroxide 1M (2×15 ml) and with water (15 ml). The organic phase was concentrated under vacuum to 15 ml and further ethyl acetate (15 ml) was added. The solution was heated to 70° C., succinic acid (0.842 g) was added, and the mixture was stirred at this temperature for 15 min. A seed (3 mg) was added. The mixture was stirred at 70° C. further 20 min and than cooled to 20° C. and stirred for two hrs. The suspension was filtered, the cake washed twice with ethyl acetate (2×3 ml) and the solid dried in a vacuum oven at 40° C. overnight, giving 3.59 g of final product. (Yield=74%).

1. A process for the preparation of 2-(methyloxy)-N-[2-methyl-1-phenyl-2-(1-pyrrolidinyl)propyl]-4,6-bis(trifluoromethyl)benzamide, which process comprises:
step (i): treatment of 2,4-ditrifluoromethyl-6-methoxy-benzoic acid with a compound of formula (III):
R₁—X (III)
wherein:
R₁ is selected from the group consisting of C₁₋₆alkylsulfonyl, arylsulphonyl and diC₁₋₆alkylphosphate diester and X is chlorine or bromine in the presence of a base and an aprotic solvent; followed by
step (ii): reaction with [2-methyl-1-phenyl-2-(1-pyrrolidinyl)propyl]amine or a salt thereof.

2. A process for the preparation of (R)-2-(methyloxy)-N-[2-methyl-1-phenyl-2-(1-pyrrolidinyl)propyl]-4,6-bis(trifluoromethyl)benzamide, which process comprises:
step (i): treatment of 2,4-ditrifluoromethyl-6-methoxy-benzoic acid with a compound of formula (III):
R₁—X (III)
wherein:
R₁ is selected from the group consisting of C₁₋₆alkylsulfonyl, arylsulphonyl and diC₁₋₆alkylphosphate diester and X is chlorine or bromine in the presence of a base and an aprotic solvent; followed by
step (ii): reaction with (R)-(+)-[2-methyl-1-phenyl-2-(1-pyrrolidinyl)propyl]amine or a salt thereof.

3. The process according to claim 1 wherein R₁ is selected from the group consisting of mesyl, tosyl and diethyl phosphate diester.

4. The process according to claim 1 wherein R₁ is n-propylsulphonyl.

5. The process according to claim 1 wherein X is chlorine.

6. The process according to claim 1 wherein the base in step (i) of the process is a tertiary amine.

7. The process according to claim 6 wherein the tertiary amine is triethylamine.

8. The process according to claim 1 wherein the aprotic solvent in step (i) of the process is selected from the group consisting of acetonitrile, methylene chloride and ethyl acetate.

9. The process according to claim 8 wherein the aprotic solvent is acetonitrile.

10. The process according to claim 8 wherein the aprotic solvent is ethyl acetate.

11. A process for the formation of a compound of formula (I): 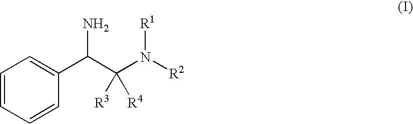
wherein:
R¹ and R² are independently selected from hydrogen and C₁₋₄alkyl, optionally substituted with one or more groups Y; or R¹ and R² together with the nitrogen atom to which they are attached form a saturated or partially unsaturated 4-, 5- 6- or 7-membered carbocyclic ring optionally substituted with a group Y′;
Y is selected from the group consisting of C₁₋₄alkoxy, hydroxy, haloC₁₋₄alkoxy and C₃₋₅cycloalkyl;
Y′ is selected from the group consisting of C₁₋₄alkyl, C₁₋₄alkoxy, halogen, hydroxy, haloC₁₋₄alkoxy, C₃₋₅cycloalkyl and C₅₋₁₀aryl or Y′ forms a —CH₂— or —CH₂—CH₂— bridge between two atoms on the 4-, 5-, 6-, or 7-membered carbocyclic ring;
R³ and R⁴ are independently C₁₋₄alkyl, optionally substituted with one or more groups X; or R³ and R⁴ together with the carbon atom to which they are attached form a saturated 5- or 6-membered carbocyclic ring optionally substituted with one or more groups X′, in the case of R³ and R⁴ together with the carbon atom to which they are attached forming a 5-membered saturated carbocyclic ring, that ring may optionally further comprise an additional heteroatom group selected from O, N and S(O)ₘ; where m=0, 1 or 2;
X is selected from the group consisting of halogen, hydroxy, C₁₋₄alkoxy, haloC₁₋₄alkyl, haloC₁₋₄alkoxy and C₅₋₁₀aryl; and
X′ is selected from the group consisting of halogen, hydroxy, C₁₋₄alkyl, C₁₋₄alkoxy, haloC₁₋₄alkoxy haloC₁₋₄alkoxy and C₅₋₁₀aryl;
whereby R¹, R², R³ and R⁴ are not all simultaneously unsubstituted methyl;
the process comprising reducing a compound of formula (II): 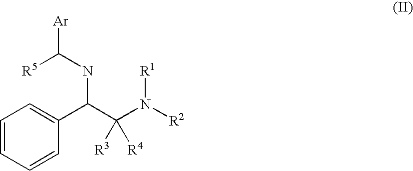
wherein R¹, R², R³ and R⁴ are as defined for formula (I), R⁵ is C₁₋₄alkyl and Ar is optionally substituted phenyl; using hydrogen and a palladium catalyst.

12. A process for the formation of 2-methyl-1-phenyl-2-(1-pyrrolidinyl)propyl]amine, the process comprising reducing a compound of formula (IIa): 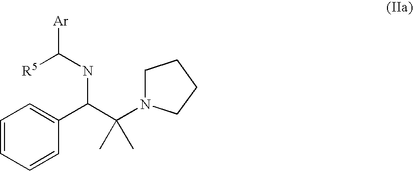
wherein R⁵ is C₁₋₄alkyl and Ar is optionally substituted phenyl; using hydrogen and a palladium catalyst.

13. A process according to claim 11 wherein the reaction takes place at an elevated temperature.

14. A process according to claim 11 wherein the reaction takes place in an alcoholic solvent.

15. A process according to claim 11 wherein the reaction takes place in the presence of an organic acid or sulphuric acid.

16. [(1R)-2-Methyl-1-phenyl-2-(1-pyrrolidinyl)propyl]-R-(+)-α-methylbenzylamine or a salt or solvate thereof:

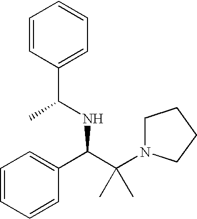

17. A process for the formation of a compound of formula (II) as defined in claim 11, comprising:
(i) reaction of a compound of formula (VIII): 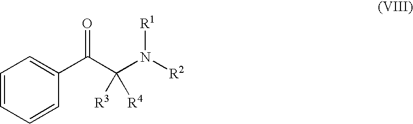
wherein R¹, R², R³ and R⁴ are as defined for formula (I) in claim 11, with a compound of formula (IV): 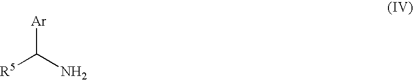
wherein R⁵ is C₁₋₄alkyl and Ar is phenyl optionally substituted by one or more groups; followed by
(ii) reduction with a sodium borohydride derivative.

18. A process for the formation of a compound of formula (IIa) as defined in claim 12, comprising:
(i) reaction of 2-pyrrolidinyl-2-methylpropiophenone: 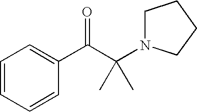
with a compound of formula (IV): 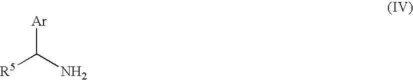
wherein R⁵ is C₁₋₄alkyl and Ar is optionally substituted phenyl; followed by
(ii) reduction with a sodium borohydride derivative.

19. A process for the formation of [(1R)-2-methyl-1-phenyl-2-(1-pyrrolidinyl)propyl] R-(+)-α-methylbenzylamine, comprising:
(i) reaction of 2-pyrrolidinyl-2-methylpropiophenone with R-(+)-α-methylbenzylamine, followed by
(ii) reduction with a sodium borohydride derivative.

20. A process according to claim 17 wherein step (i) is carried out in an aprotic solvent in the presence of titanium (IV) chloride and a tertiary base.

21. A process according to claim 17 wherein step (i) is carried out at elevated temperature.

22. A process for the formation of a compound of formula (VIII) as defined in claim 17, comprising treatment of a compound of formula (V): 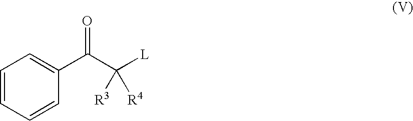
wherein R³ and R⁴ are as defined for formula (I) in claim 11 and L is a leaving group, with a compound of formula (VI):
R⁶—OH (VI)
wherein R⁶ is C1-4alkyl, in the presence of a base, followed by reaction with a compound of formula (VII):
NHR¹R² (VII)
wherein R¹ and R² are as defined for formula (I) in claim 11.

23. A process for the formation of 2-pyrrolidinyl-2-methylpropiophenone, comprising treatment of a compound of formula (Va): 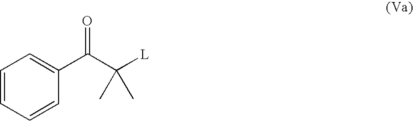
wherein L is a leaving group, with a compound of formula (VI):
R⁶—OH (VI)
wherein R⁶ is C1-4alkyl, in the presence of a base, followed by reaction with pyrrolidine.

24. 2-pyrrolidinyl-2-methylpropiophenone or a salt or solvate thereof:
